(19) United States
(12) Patent Application Publication
(10) Pub. No.: US 2021/0085838 A1
(43) Pub. Date: Mar. 25, 2021

(54) WOUND THERAPY SYSTEM WITH FLUID INSTILLATION AND REMOVAL

(71) Applicant: KCI LICENSING, INC., San Antonio, TX (US)

(72) Inventors: Christopher B. LOCKE, Bournemouth (GB); David G. WHYTE, Wareham (GB)

(21) Appl. No.: 16/630,575

(22) PCT Filed: Jul. 25, 2018
(86) PCT No.: PCT/US2018/043620

§ 371 Date: Jan. 13, 2020

Related U.S. Application Data
(60) Provisional application No. 62/537,550, filed on Jul. 27, 2017.

(51) Int. Cl. A61M 1/00; A61M 39/22; A61M 39/24

(52) U.S. Cl. CPC A61M 1/009; A61M 1/0084; A61M 1/0001; A61M 2205/502; A61M 39/24; A61M 2205/3344; A61M 39/223

(57) ABSTRACT

A wound therapy system is configured to deliver fluid to a wound site and remove fluid from the wound site. The wound therapy system includes a combined fluid delivery and removal line fluidly coupled to the wound site and a peristaltic pump coupled to the combined fluid delivery and removal line. The peristaltic pump is configured to operate in a first direction to deliver instillation fluid to the wound site via the combined fluid delivery and removal line and operate in a second direction opposite the first direction to remove fluid from the wound site via the combined fluid delivery and removal line.

### CROSS-REFERENCE TO RELATED APPLICATION

This application claims the benefit of priority to U.S. Provisional Application No. 62/537,550, filed on Jul. 27, 2017, which is incorporated herein by reference in its entirety.

### BACKGROUND

The present disclosure relates generally to a wound therapy system, and more particularly to a wound therapy system configured to deliver fluid to a wound and remove fluid from the wound.

Negative pressure wound therapy (NPWT) is a type of wound therapy that involves applying a negative pressure to a wound site to promote wound healing. Some wound treatment systems apply negative pressure to a wound using a pneumatic pump to generate the negative pressure and flow required. Recent advancements in wound healing with NPWT involve applying topical fluids to wounds to work in combination with NPWT. The fluids provided to the wound are typically gravity fed. However, this does not allow the system or the user to accurately know and control the quantity of fluid delivered to the wound.

Recent developments to overcome this challenge have involved adding a fluid pump to the system which can control the delivery of fluids to the wound. Accordingly, combined fluid delivery and removal systems typically include one pneumatic pump and one fluid pump, each of which are connected to the wound via different tubes. The resulting therapy system is often large and complex to setup. It would be desirable to provide a wound therapy system that overcomes these and other disadvantages of conventional fluid delivery and removal systems

### SUMMARY

One implementation of the present disclosure is a wound therapy system for delivering fluid to a wound site and removing fluid from the wound site. The wound therapy system includes an instillation fluid container configured to store instillation fluid for delivery to the wound site, a removed fluid container configured to store fluid removed from the wound site, a combined fluid delivery and removal line, and a single pump. The combined fluid delivery and removal line is fluidly coupled to the wound site, the instillation fluid container, and the removed fluid container. The pump is coupled to the combined fluid delivery and removal line. The pump is configured to operate in a first direction to draw the instillation fluid from the instillation fluid container and deliver the instillation fluid to the wound site via the combined fluid delivery and removal line. The pump is configured to operate in a second direction opposite the first direction to remove fluid from the wound site and deliver the removed fluid to the removed fluid container via the combined fluid delivery and removal line.

In some embodiments, the pump is a peristaltic pump and the combined fluid delivery and removal line passes through the peristaltic pump.

In some embodiments, the wound therapy system includes a three-way valve fluidly coupled to the instillation fluid container, the removed fluid container, and the combined fluid delivery and removal line. In some embodiments, the three-way valve is configured to direct fluid flow from the instillation fluid container to the combined fluid delivery and removal line when the pump is operated in the first direction and direct fluid flow from the combined fluid delivery and removal line to the removed fluid container when the pump is operated in the second direction.

In some embodiments, the three-way valve includes a first one-way valve and a second one-way valve. The first one-way valve can be configured to allow fluid flow from the instillation fluid container to the combined fluid delivery and removal line and prevent fluid flow from the combined fluid delivery and removal line to the instillation fluid container. The second one-way valve can be configured to allow fluid flow from the combined fluid delivery and removal line to the removed fluid container and prevent fluid flow from the removed fluid container to the combined fluid delivery and removal line.

In some embodiments, the three-way valve includes a first port fluidly coupled to the instillation fluid container, a second port fluidly coupled to the removed fluid container, and a third port fluidly coupled to the combined fluid delivery and removal line. In some embodiments, the wound therapy system includes an instillation fluid line connecting the first port of the three-way valve to the instillation fluid container and a removed fluid line connecting the second port of the three-way valve to the removed fluid container.

In some embodiments, the wound therapy system includes an instillation fluid line connecting the instillation fluid container to the combined fluid delivery and removal line and a one-way valve disposed along the instillation fluid line. The one-way valve can be configured to allow fluid flow from the instillation fluid container to the combined fluid delivery and removal line and prevent fluid flow from the combined fluid delivery and removal line to the instillation fluid container.

In some embodiments, the wound therapy system includes a removed fluid line connecting the removed fluid container to the combined fluid delivery and removal line and a one-way valve disposed along the removed fluid line. The one-way valve can be configured to allow fluid flow from the combined fluid delivery and removal line to the removed fluid container and prevent fluid flow from the removed fluid container to the combined fluid delivery and removal line.

In some embodiments, the wound therapy system includes a pressure sensor configured to measure pressure at the wound site and a controller configured to operate the pump based on the measured pressure. In some embodiments, the controller is configured to monitor the measured pressure at the wound site and operate the pump to maintain the measured pressure at a predetermined pressure threshold.

In some embodiments, the controller is configured to monitor the measured pressure at the wound site, operate the pump in the first direction to deliver the instillation fluid to the wound site in response to a determination that the measured pressure is less than a positive pressure threshold, and stop operating the pump in the first direction in response to a determination that the measured pressure is equal to or greater than the positive pressure threshold.

In some embodiments, the controller is configured to monitor the measured pressure at the wound site, operate the pump in the second direction to remove fluid from the wound site in response to a determination that the measured pressure is equal to or greater than a negative pressure threshold, stop operating the pump in the second direction in response to a determination that the measured pressure is less than the negative pressure threshold.

In some embodiments, the controller is configured to estimate a volume of the wound site by counting a number of rotations of the pump required to change the measured pressure by a predetermined amount and estimating the volume of the wound site based on the counted number of rotations.

In some embodiments, counting the number of rotations of the pump required to change the measured pressure by a predetermined amount includes operating the pump in the first direction until the measured pressure reaches a positive pressure threshold, operating the pump in the second direction until the measured pressure reaches a negative pressure threshold, and counting the number of rotations of the pump required to change the measured pressure from the positive pressure threshold to the negative pressure threshold.

Another implementation of the present disclosure is a wound therapy system for delivering fluid to a wound site and removing fluid from the wound site. The wound therapy system includes a combined fluid delivery and removal line fluidly coupled to the wound site and a peristaltic pump coupled to the combined fluid delivery and removal line. The peristaltic pump is configured to operate in a first direction to deliver instillation fluid to the wound site via the combined fluid delivery and removal line and operate in a second direction opposite the first direction to remove fluid from the wound site via the combined fluid delivery and removal line.

In some embodiments, the wound therapy system includes an instillation fluid container configured to store the instillation fluid for delivery to the wound site. The peristaltic pump may be configured to draw the instillation fluid from the instillation fluid container and deliver the instillation fluid to the wound site via the combined fluid delivery and removal line when the peristaltic pump is operated in the first direction.

In some embodiments, the wound therapy system includes an instillation fluid line connecting the instillation fluid container and the combined fluid delivery and removal line. The wound therapy system may include a first one-way valve disposed along the instillation fluid line. The first one-way valve may be configured to allow fluid flow from the instillation fluid container to the combined fluid delivery and removal line and prevent fluid flow from the combined fluid delivery and removal line to the instillation fluid container.

In some embodiments, the wound therapy system includes a removed fluid container configured to store the fluid removed from the wound site. The peristaltic pump may be configured to draw the removed fluid from the wound site and deliver the removed fluid to the removed fluid container via the combined fluid delivery and removal line when the peristaltic pump is operated in the second direction.

In some embodiments, the wound therapy system includes a removed fluid line connecting the removed fluid container and the combined fluid delivery and removal line. The wound therapy system may include a second one-way valve disposed along the removed fluid line. The second one-way valve may be configured to allow fluid flow from the combined fluid delivery and removal line to the removed fluid container and prevent fluid flow from the removed fluid container to the combined fluid delivery and removal line.

In some embodiments, the wound therapy system includes an instillation fluid container, a removed fluid container, and a three-way valve fluidly coupled to the instillation fluid container, the removed fluid container, and the combined fluid delivery and removal line.

In some embodiments, the three-way valve is configured to direct fluid flow from the instillation fluid container to the combined fluid delivery and removal line when the peristaltic pump is operated in the first direction and direct fluid flow from the combined fluid delivery and removal line to the removed fluid container when the peristaltic pump is operated in the second direction.

In some embodiments, the three-way valve includes a first one-way valve and a second one-way valve. The first one-way valve can be configured to allow fluid flow from the instillation fluid container to the combined fluid delivery and removal line and prevent fluid flow from the combined fluid delivery and removal line to the instillation fluid container. The second one-way valve can be configured to allow fluid flow from the combined fluid delivery and removal line to the removed fluid container and prevent fluid flow from the removed fluid container to the combined fluid delivery and removal line.

In some embodiments, the three-way valve includes a first port fluidly coupled to the instillation fluid container, a second port fluidly coupled to the removed fluid container, and a third port fluidly coupled to the combined fluid delivery and removal line.

In some embodiments, the wound therapy system includes an instillation fluid line connecting the first port of the three-way valve to the instillation fluid container and a removed fluid line connecting the second port of the three-way valve to the removed fluid container.

In some embodiments, the wound therapy system includes a pressure sensor configured to measure pressure at the wound site and a controller configured to operate the peristaltic pump based on the measured pressure. In some embodiments, the controller is configured to monitor the measured pressure at the wound site and operate the peristaltic pump to maintain the measured pressure at a predetermined pressure threshold.

In some embodiments, the controller is configured to monitor the measured pressure at the wound site, operate the peristaltic pump in the first direction to deliver the instillation fluid to the wound site in response to a determination that the measured pressure is less than a positive pressure threshold, and stop operating the peristaltic pump in the first direction in response to a determination that the measured pressure is greater than or equal to the positive pressure threshold.

In some embodiments, the controller is configured to monitor the measured pressure at the wound site, operate the peristaltic pump in the second direction to remove fluid from the wound site in response to a determination that the measured pressure is greater than or equal to a negative pressure threshold, and stop operating the peristaltic pump in the second direction in response to a determination that the measured pressure is less than the negative pressure threshold.

In some embodiments, the controller is configured to estimate a volume of the wound site by counting a number of rotations of the peristaltic pump required to change the measured pressure by a predetermined amount and estimating the volume of the wound site based on the counted number of rotations.

In some embodiments, counting the number of rotations of the peristaltic pump required to change the measured pressure by a predetermined amount includes operating the peristaltic pump in the first direction until the measured pressure reaches a positive pressure threshold, operating the peristaltic pump in the second direction until the measured pressure reaches a negative pressure threshold, counting the number of rotations of the peristaltic pump required to change the measured pressure from the positive pressure threshold to the negative pressure threshold.

Another implementation of the present disclosure is a method for delivering fluid to a wound site and removing fluid from the wound site. The method includes fluidly coupling a combined fluid delivery and removal line to the wound site, operating a peristaltic pump in a first direction to deliver instillation fluid to the wound site via the combined fluid delivery and removal line, and operating the peristaltic pump in a second direction opposite the first direction to remove fluid from the wound site via the combined fluid delivery and removal line.

In some embodiments, operating the peristaltic pump in the first direction includes drawing the instillation fluid from an instillation fluid container and delivering the instillation fluid from the instillation fluid container to the wound site via the combined fluid delivery and removal line.

In some embodiments, the method includes using a first one-way valve to allow fluid flow from the instillation fluid container to the combined fluid delivery and removal line and prevent fluid flow from the combined fluid delivery and removal line to the instillation fluid container.

In some embodiments, operating the peristaltic pump in the second direction includes drawing the removed fluid from the wound site and delivering the removed fluid from the wound site to a removed fluid container via the combined fluid delivery and removal line.

In some embodiments, the method includes using a second one-way valve to allow fluid flow from the combined fluid delivery and removal line to the removed fluid container and prevent fluid flow from the removed fluid container to the combined fluid delivery and removal line.

In some embodiments, the method includes using a three-way valve to control fluid flow among an instillation fluid container, a removed fluid container, and the combined fluid delivery and removal line.

In some embodiments, using the three-way valve to control fluid flow includes directing fluid flow from the instillation fluid container to the combined fluid delivery and removal line when the peristaltic pump is operated in the first direction and directing fluid flow from the combined fluid delivery and removal line to the removed fluid container when the peristaltic pump is operated in the second direction.

In some embodiments, the three-way valve includes a first port fluidly coupled to the instillation fluid container, a second port fluidly coupled to the removed fluid container, and a third port fluidly coupled to the combined fluid delivery and removal line.

In some embodiments, operating the peristaltic pump in the first direction causes the instillation fluid to flow through an instillation fluid line connecting the first port of the three-way valve to the instillation fluid container. In some embodiments, operating the peristaltic pump in the second direction causes the removed fluid to flow through a removed fluid line connecting the second port of the three-way valve to the removed fluid container.

In some embodiments, the method includes measuring a pressure at the wound site and operating the peristaltic pump based on the measured pressure. In some embodiments, operating the peristaltic pump based on the measured pressure includes operating the peristaltic pump to maintain the measured pressure at a predetermined pressure threshold.

In some embodiments, operating the peristaltic pump based on the measured pressure includes operating the peristaltic pump in the first direction to deliver the instillation fluid to the wound site in response to a determination that the measured pressure is less than a positive pressure threshold and stopping operating of the peristaltic pump in the first direction in response to a determination that the measured pressure is greater than or equal to the positive pressure threshold.

In some embodiments, operating the peristaltic pump based on the measured pressure includes operating the peristaltic pump in the second direction to remove fluid from the wound site in response to a determination that the measured pressure is greater than or equal to a negative pressure threshold and stopping operation of the peristaltic pump in the second direction in response to a determination that the measured pressure is less than the negative pressure threshold.

In some embodiments, the method includes counting a number of rotations of the peristaltic pump required to change the measured pressure by a predetermined amount and estimating the volume of the wound site based on the counted number of rotations.

In some embodiments, counting the number of rotations of the peristaltic pump required to change the measured pressure by a predetermined amount includes operating the peristaltic pump in the first direction until the measured pressure reaches a positive pressure threshold, operating the peristaltic pump in the second direction until the measured pressure reaches a negative pressure threshold, and counting the number of rotations of the peristaltic pump required to change the measured pressure from the positive pressure threshold to the negative pressure threshold.

Those skilled in the art will appreciate that the summary is illustrative only and is not intended to be in any way limiting. Other aspects, inventive features, and advantages of the devices and/or processes described herein, as defined solely by the claims, will become apparent in the detailed description set forth herein and taken in conjunction with the accompanying drawings.

### BRIEF DESCRIPTION OF THE DRAWINGS

FIG. 1 is a block diagram of a wound therapy system including an instillation fluid container, a removed fluid container, a valve, and a pump, according to an exemplary embodiment.

FIG. 2 is a block diagram illustrating the valve of FIG. 1 in greater detail, according to an exemplary embodiment.

FIG. 3 is a block diagram illustrating operation of the wound therapy system of FIG. 1 during fluid instillation, according to an exemplary embodiment.

FIG. 4 is a block diagram illustrating operation of the wound therapy system of FIG. 1 during fluid removal, according to an exemplary embodiment.

FIG. 5 is a block diagram of the wound therapy system of FIG. 1 with an additional therapy device, according to an exemplary embodiment.

FIG. 6 is a block diagram illustrating operation of the wound therapy system of FIG. 1 when inflating a wound site with air, according to an exemplary embodiment.

FIG. 7 is a block diagram illustrating operation of the wound therapy system of FIG. 1 when removing fluid from the wound site, according to an exemplary embodiment.

FIG. 8 is a block diagram illustrating the therapy device detecting and indicating the level of instillation fluid in the instillation fluid container, according to an exemplary embodiment.

FIG. 9 is a block diagram illustrating the therapy device detecting and indicating the level of removed fluid in the removed fluid container, according to an exemplary embodiment.

FIG. 10 is a block diagram illustrating the therapy device in greater detail, according to an exemplary embodiment.

### DETAILED DESCRIPTION

### Overview

Referring generally to the FIGURES, a wound therapy system with fluid instillation and removal and components thereof are shown, according to various exemplary embodiments. The wound therapy system may include an instillation fluid container, a removed fluid container, and a pump. The instillation fluid container can be configured to store an instillation fluid (e.g., a cleansing fluid, a prescribed fluid, etc.) for delivery to a wound site. The removed fluid container can be configured to store a fluid removed from the wound site (e.g., wound exudate, previously-delivered instillation fluid, etc.). Both the instillation fluid container and the removed fluid container may be fluidly coupled to the wound site via a combined fluid delivery and removal line.

In some embodiments, the wound therapy system includes one-way valves. The one-way valves prevent fluid flow into the instillation fluid container and out of the removed fluid container. For example, a first one-way valve can be configured to allow fluid flow from the instillation fluid container to the combined fluid delivery and removal line and prevent fluid flow from the combined fluid delivery and removal line to the instillation fluid container. A second one-way valve can be configured to allow fluid flow from the combined fluid delivery and removal line to the removed fluid container and prevent fluid flow from the removed fluid container to the combined fluid delivery and removal line.

The pump can be operated in a first direction to draw the instillation fluid from the instillation fluid container and deliver the instillation fluid to the wound site via the combined fluid delivery and removal line. The pump can be operated in a second direction opposite the first direction to remove fluid from the wound site and deliver the removed fluid to the removed fluid container via the combined fluid delivery and removal line. Advantageously, a single pump can be used to both deliver the instillation fluid to the wound site and remove the removed fluid from the wound site. The pump can also be operated to establish a negative pressure at the wound site (relative to atmospheric pressure) to provide negative pressure wound therapy (NPWT).

In some embodiments, the wound therapy system is configured to estimate the volume of the wound site. The estimated volume can be used to ensure the correct quantity of fluid is delivered during therapy. The wound therapy system can track wound volume over time to determine a change in wound volume during the progression of healing. Additional features and advantages of the wound therapy system are described in detail below.

### Wound Therapy System

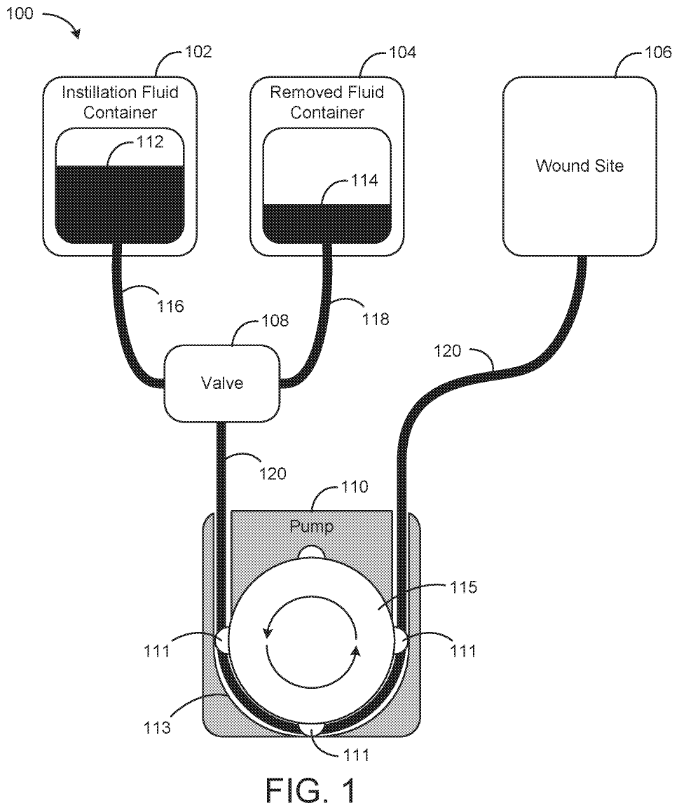

Referring now to FIG. 1, a wound therapy system 100 is shown, according to an exemplary embodiment. Wound therapy system 100 is shown to include an instillation fluid container 102, a removed fluid container 104, a valve 108, and a pump 110. Instillation fluid container 102 can be configured to store an instillation fluid 112 for delivery to a wound site 106. Instillation fluid 112 can include, for example, a cleansing fluid, a prescribed fluid, a medicated fluid, an antibiotic fluid, or any other type of fluid which can be delivered to wound site 106 during wound treatment. Instillation fluid container 102 may be fluidly connected to valve 108 via an instillation fluid line 116. Instillation fluid 112 may flow from instillation fluid container 102 to valve 108 through instillation fluid line 116 when system 100 is operated in a fluid instillation mode.

Removed fluid container 104 can be configured to store a fluid 114 removed from wound site 106 (i.e., removed fluid 114). Removed fluid 114 can include, for example, wound exudate (e.g., bodily fluids), air, instillation fluid 112 previously delivered to wound site 106, or any other type of fluid which can be removed from wound site 106 during wound treatment. Removed fluid container 104 may be fluidly connected to valve 108 via a removed fluid line 118. Removed fluid 114 may flow from valve 108 to removed fluid container 104 through removed fluid line 118 when system 100 is operated in a fluid removal mode.

In some embodiments, valve 108 is fluidly connected to wound site 106 via a combined fluid delivery and removal line 120. Combined fluid delivery and removal line 120 may be fluidly coupled to wound site 106, instillation fluid container 102 (via instillation fluid line 116), and removed fluid container 104 (via removed fluid line 118). Combined fluid delivery and removal line 120 can be configured to transport both instillation fluid 112 and removed fluid 114. For example, instillation fluid 112 can flow from valve 108 to wound site 106 via combined fluid delivery and removal line 120 when system 100 is operated in the fluid instillation mode. Similarly, removed fluid 114 can flow from wound site 106 to valve 108 via combined fluid delivery and removal line 120 when system 100 is operated in the fluid removal mode. In some embodiments, combined fluid delivery and removal line 120 passes through pump 110.

Pump 110 can be operated to cause fluid flow in system 100. During fluid instillation, pump 110 can operate in a first direction (e.g., counterclockwise in FIG. 1) to draw instillation fluid 112 from instillation fluid container 102 and deliver instillation fluid 112 to wound site 106 via combined fluid delivery and removal line 120. During fluid removal, pump 110 can operate in a second direction opposite the first direction (e.g., clockwise in FIG. 1) to remove fluid 114 from wound site 106 and deliver removed fluid 114 to removed fluid container 104 via combined fluid delivery and removal line 120. In some embodiments, the operation of pump 110 is controlled by a controller or therapy device, described in greater detail below.

In some embodiments, pump 110 is a peristaltic pump having a rotor 115 and plurality of head rollers 111. Rotor 115 can be configured to rotate in a first direction (e.g., counterclockwise in FIG. 1) during fluid instillation and in a second direction opposite the first direction (e.g., clockwise in FIG. 1) during fluid removal. Head rollers 111 can be configured to seal combined fluid delivery and removal line 120 against an internal surface 113 of pump 110 (e.g., by pinching combined fluid delivery and removal line 120 between surface 113 and head rollers 111). When rotor 115 rotates, head rollers 111 may move along the external surface of combined fluid delivery and removal line 120, thereby causing fluid flow through combined fluid delivery and removal line 120 via peristaltic action. Although pump 110 is shown as a peristaltic pump, it is contemplated that pump 110 can be any type of pump (e.g., peristaltic, non-peristaltic, etc.) configured to cause fluid flow in system 100.

### Valve Configuration

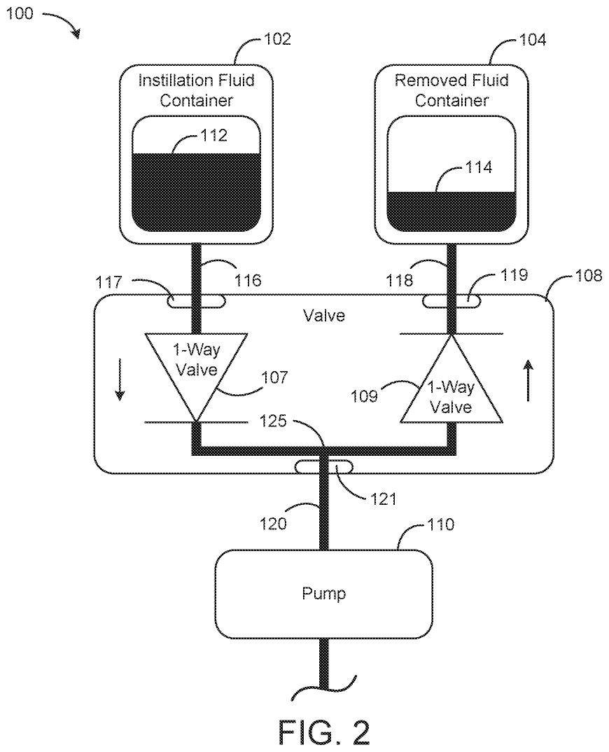

Referring now to FIG. 2, valve 108 is shown in greater detail, according to an exemplary embodiment. In some embodiments, valve 108 is a three-way valve having a first port 117, a second port 119, and a third port 121. First port 117 may be fluidly coupled to instillation fluid container 102 via instillation fluid line 116. Second port 119 may be fluidly coupled to removed fluid container 104 via removed fluid line 118. Third port 121 may be fluidly coupled to pump 110 and/or wound site 106 via combined fluid delivery and removal line 120. In some embodiments, instillation fluid line 116, removed fluid line 118, and combined fluid delivery and removal line 120 intersect at a three-way junction 125 within valve 108.

Valve 108 is shown to include a first one-way valve 107 and a second one-way valve 109. One-way valve 107 can be fluidly connected to both first port 117 and third port 121 such that instillation fluid 112 flows through one-way valve 107 when system 100 is operated in the fluid instillation mode. One-way valve 107 can be configured to allow fluid flow from instillation fluid container 102 to combined fluid delivery and removal line 120 and configured to prevent fluid flow from combined fluid delivery and removal line 120 to instillation fluid container. Accordingly, one-way valve 107 may permit instillation fluid 112 to flow from instillation fluid container 102 to combined fluid delivery and removal line 120, and may prevent removed fluid 114 from flowing from combined fluid delivery and removal line 120 to instillation fluid container 102.

One-way valve 109 can be fluidly connected to both second port 119 and third port 121 such that removed fluid 114 flows through one-way valve 109 when system 100 is operated in the fluid removal mode. One-way valve 109 can be configured to allow fluid flow from combined fluid delivery and removal line 120 to removed fluid container 104 and configured to prevent fluid flow from removed fluid container 104 to combined fluid delivery and removal line 120. Accordingly, one-way valve 109 may permit removed fluid 114 to flow from combined fluid delivery and removal line 120 to removed fluid container 104, and may prevent removed fluid 114 from flowing from removed fluid container 104 to combined fluid delivery and removal line 120.

### Fluid Instillation and Removal

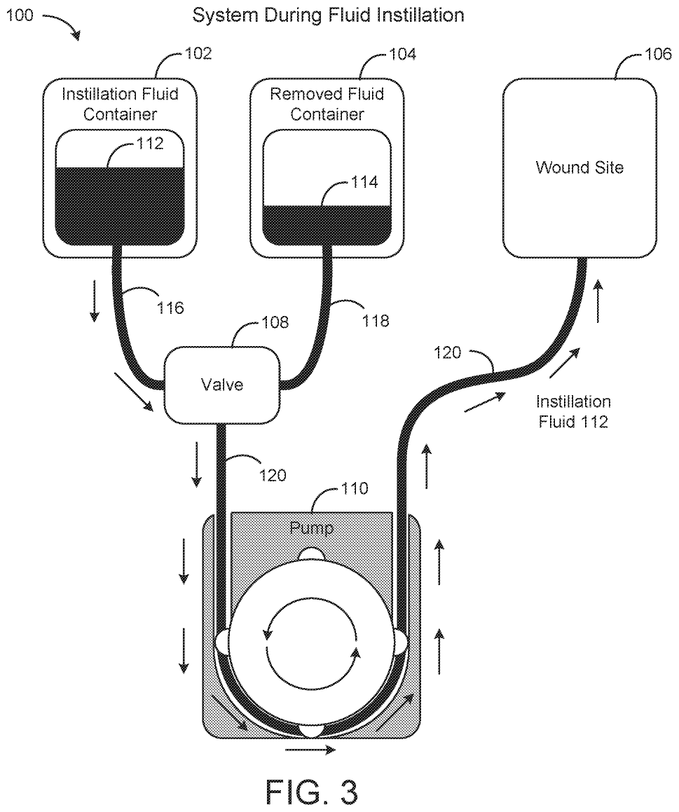

Referring now to FIG. 3, the operation of system 100 during fluid instillation is shown, according to an exemplary embodiment. FIG. 3 illustrates the functionality of system 100 when operated in the fluid instillation mode. During fluid instillation, pump 110 can be operated in a first direction (e.g., counterclockwise in FIG. 3). The operation of pump 110 causes instillation fluid 112 to flow along the path indicated by the arrows in FIG. 3. For example, instillation fluid 112 can be drawn from instillation fluid container 102 via instillation fluid line 116 and may enter valve 108. One-way valve 107 within valve 108 allows instillation fluid 112 to flow through valve 108 and into combined fluid delivery and removal line 120. Instillation fluid 112 then flows through combined fluid delivery and removal line 120, through pump 110, and is delivered to wound site 106. One-way valve 109 within valve 108 prevents removed fluid 114 from being drawn from removed fluid container 104 via removed fluid line 118.

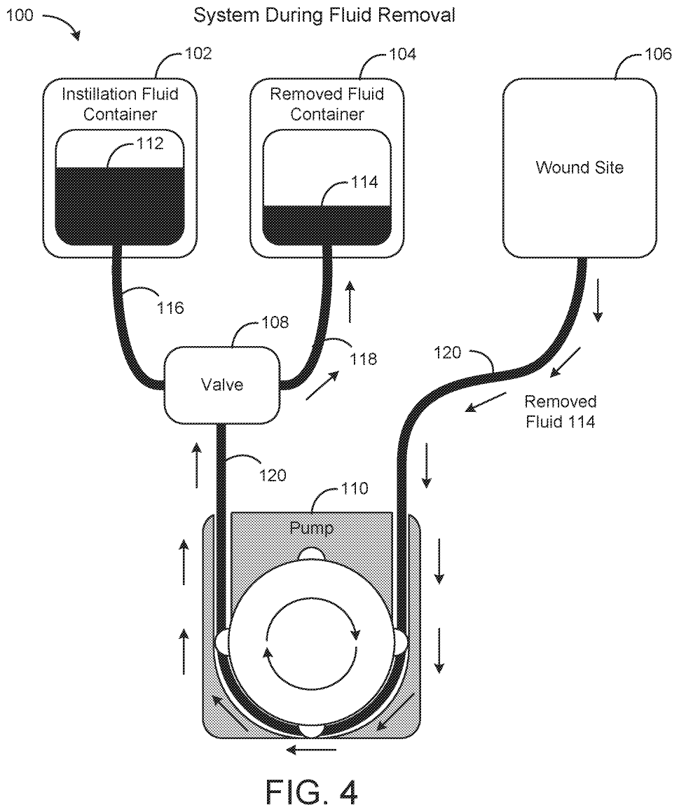

Referring now to FIG. 4, the operation of system 100 during fluid removal is shown, according to an exemplary embodiment. FIG. 4 illustrates the functionality of system 100 when operated in the fluid removal mode. During fluid removal, pump 110 can be operated in a second direction (e.g., clockwise in FIG. 4). The operation of pump 110 causes removed fluid 114 to flow along the path indicated by the arrows in FIG. 4. For example, removed fluid 114 can be drawn from wound site 106 via combined fluid delivery and removal line 120, through pump 110, and delivered to valve 108. One-way valve 109 within valve 108 allows removed fluid 114 to flow through valve 108 and into removed fluid line 118. Removed fluid 114 is then delivered to removed fluid container 104 via removed fluid line 118. One-way valve 107 within valve 108 prevents removed fluid 114 from flowing into instillation fluid line 116.

### Wound Therapy System With Therapy Device

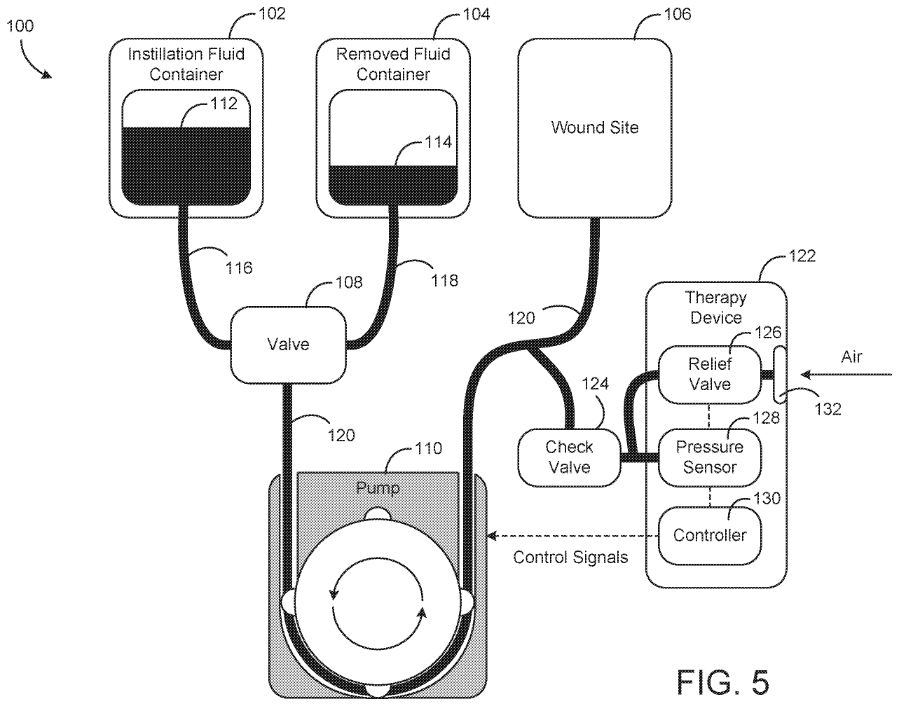

Referring now to FIG. 5, wound therapy system 100 is shown to include a therapy device 122. Therapy device 122 may be an electronic device configured to monitor conditions within system 100 and control pump 110 based on the monitored conditions. For example, therapy device 122 is shown to include a pressure sensor 128, a controller 130, and a relief valve 126. Pressure sensor 128 can be configured to measure the pressure within combined fluid delivery and removal line 120 and can send the measured pressure values to controller 130. Controller 130 can use the pressure values recorded by pressure sensor 128 to monitor the pressure within combined fluid delivery and removal line 120.

In some embodiments, wound site 106 includes a wound dressing that provides an airtight seal over a wound. Combined fluid delivery and removal line 120 can be fluidly connected to the internal volume of wound site 106 between the wound and the wound dressing. The pressure within combined fluid delivery and removal line 120 may be equivalent to the pressure at wound site 106. Accordingly, controller 130 can be configured to monitor the pressure at wound site 106 using the pressure values recorded by pressure sensor 128.

Controller 130 can be configured to operate pump 110 based on the measured pressure values. In some embodiments, controller 130 is configured to provide negative pressure wound therapy (NPWT) by maintaining the pressure at wound site 106 at a predetermined negative pressure threshold (e.g., −10 mmHg). For example, controller 130 can operate pump 110 to remove fluid from wound site 106 in response to a determination that the measured pressure is greater than the negative pressure threshold. By operating pump 110, fluid can be removed from wound site 106 and the pressure at wound site 106 may drop until the negative pressure threshold is reached.

In some embodiments, controller 130 is configured to operate pump 110 in a first direction (e.g., counterclockwise in FIG. 5) to deliver instillation fluid 112 to wound site 106 in response to a determination that the measured pressure is less than a positive pressure threshold. In some embodiments, the positive pressure threshold is a pressure value greater than atmospheric or ambient pressure around system 100. Controller 130 can be configured to stop operating pump 110 in the first direction in response to a determination that the measured pressure is equal to or greater than the positive pressure threshold. Similarly, controller 130 can be configured to operate pump 110 in a second direction (e.g., clockwise in FIG. 5) to remove fluid from wound site 106 in response to a determination that the measured pressure is equal to or greater than a negative pressure threshold. In some embodiments, the negative pressure threshold is a pressure value less than atmospheric or ambient pressure around system 100. Controller 130 can be configured to stop operating pump 110 in the second direction in response to a determination that the measured pressure is less than the negative pressure threshold.

In some embodiments, pressure sensor 128 is fluidly coupled to combined fluid delivery and removal line 120 via a check valve 124 (e.g., a one-way valve). Check valve 124 can be configured to allow fluid flow from therapy device 122 to combined fluid delivery and removal line 120 and configured to prevent fluid flow from combined fluid delivery and removal line 120 to therapy device 122. Check valve 124 may help maintain pressure sensor 128 in a clean and operable condition by preventing fluid from combined fluid delivery and removal line 120 from entering pressure sensor 128 and disrupting the operation of pressure sensor 128.

In some embodiments, relief valve 126 is fluidly connected to combined fluid delivery and removal line 120. Relief valve 126 can be configured to allow air from the atmosphere around therapy device 122 to enter system 100 via vent 132. The air can enter combined fluid delivery and removal line 120 via check valve 124 and can flow to wound site 106. Relief valve 126 can be configured to allow air to enter system 100 when the pressure at wound site 106 is below a minimum pressure threshold to prevent the pressure at wound site 106 from dropping below the minimum pressure threshold. Similarly, relief valve 126 can be configured to reduce the pressure at wound site 106 by venting fluid from within combined fluid delivery and removal line 120 when the pressure at wound site 106 is above a maximum pressure threshold.

### Wound Volume Estimation

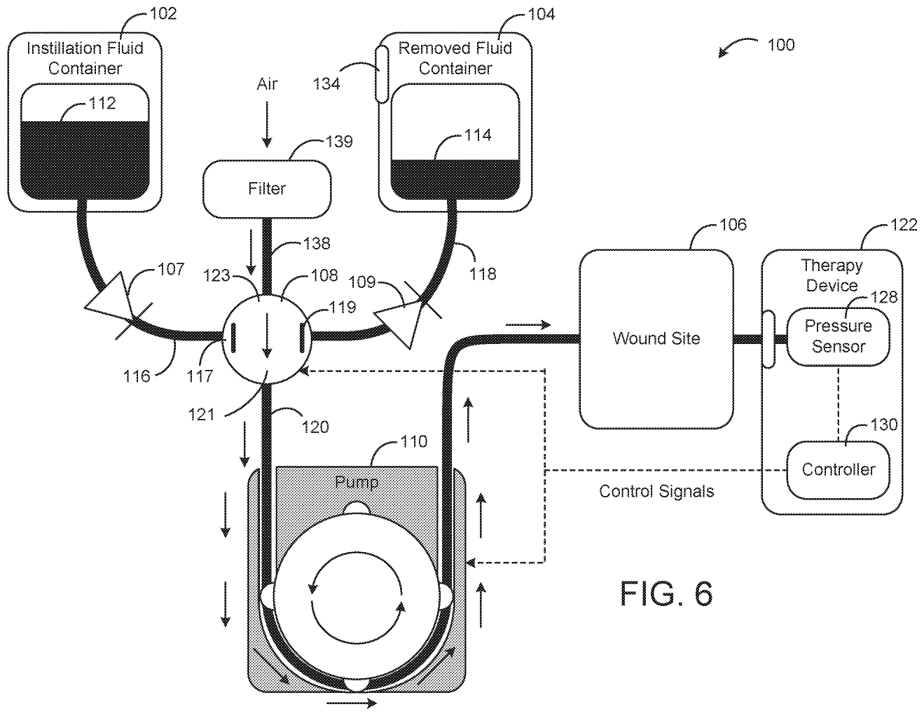

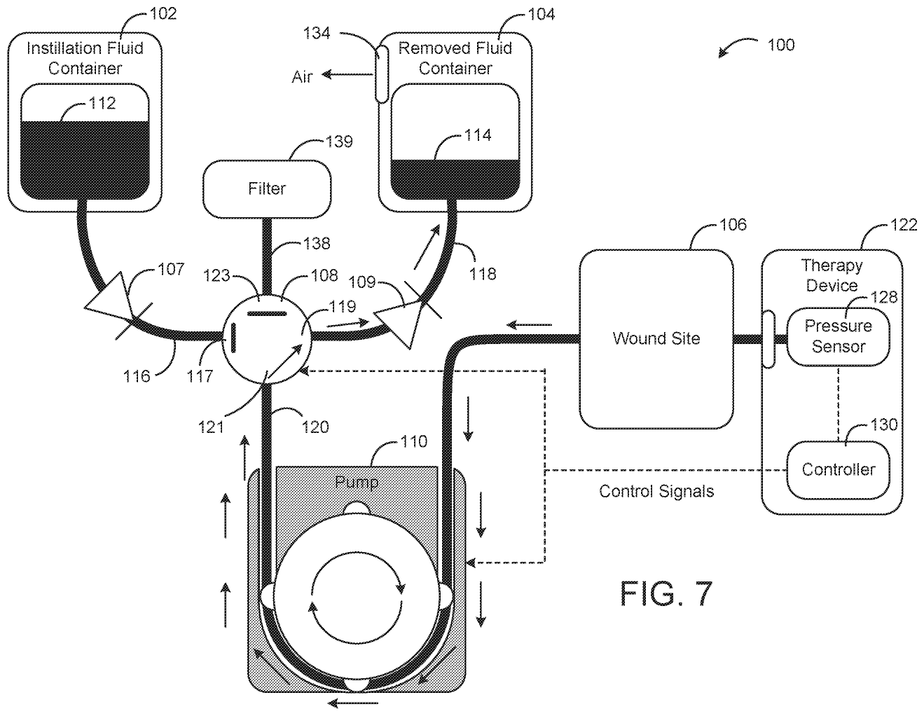

Referring now to FIGS. 6-7, wound therapy system 100 and therapy device 122 are shown, according to another exemplary embodiment. Valve 108 is shown as a controllable valve having a plurality of ports (e.g., four ports). A first port 117 of valve 108 may be fluidly coupled to instillation fluid container 102 via instillation fluid line 116. A second port 119 of valve 108 may be fluidly coupled to removed fluid container 104 via removed fluid line 118. A third port 121 of valve 108 may be fluidly coupled to wound site 106 via combined fluid delivery and removal line 120. A fourth port 123 of valve 108 may be fluidly coupled to a filter 139 via an air intake line 138. Valve 108 can be operated by controller 130 to select which of the ports 117, 119, 121, and 123 are open and closed. In other words, controller 130 can be configured to operate valve 108 to open and close ports 117, 119, 121, and 123.

In some embodiments, one-way valve 107 is positioned along instillation fluid line 116 between valve 108 and instillation fluid container 102. Similarly, one-way valve 109 can be positioned along removed fluid line 118 between valve 108 and removed fluid container 104. It is contemplated that one-way valves 107 and 109 may be part of valve 108 or separate from valve 108 in various embodiments.

Therapy device 122 is shown to include a pressure sensor 128 and a controller 130. Pressure sensor 128 can be fluidly coupled to wound site 106 and configured to measure the pressure at wound site 106. In some embodiments, wound site 106 includes a wound dressing that provides an airtight seal over a wound. Controller 130 can be configured to operate pump 110 and valve 108 based on the measured pressure to provide negative pressure wound therapy (NPWT) at wound site 106, as previously described.

In some embodiments, controller 130 is configured to estimate the volume of wound site 106. Estimating the volume of wound site 106 may involve inflating wound site 106 with air (or another fluid) until a positive pressure threshold is reached and then removing the air (or other fluid) from wound site 106 until a negative pressure threshold is reached. The number of rotations of pump 110 required to move from the positive pressure threshold to the negative pressure threshold may be proportional to the volume of wound site 106. The wound volume estimation procedure is illustrated in FIGS. 6-7.

Referring particularly to FIG. 6, the operation of system 100 to inflate wound site 106 with air is shown. Controller 130 can operate valve 108 to open ports 121 and 123 and close ports 117 and 119. This allows for airflow flow between filter 139 and wound site 106 and prevents fluid flow to/from instillation fluid container 102 and removed fluid container 104. With ports 121 and 123 open, controller 130 can operate pump 110 in a first direction (e.g., counterclockwise in FIG. 6) to draw air into air intake line 138 via filter 139. The air may flow through valve 108, through combined fluid delivery and removal line 120, and into wound site 106. Controller 130 may continue to operate pump 110 in the first direction until the pressure measured by pressure sensor 128 (i.e., the pressure at wound site 106) reaches a positive pressure threshold (e.g., +10 mmHg). When the positive pressure threshold is reached, controller 130 may stop pump 110.

Referring particularly to FIG. 7, the operation of system 100 to deflate wound site 106 is shown. Controller 130 can operate valve 108 to open ports 121 and 119 and close ports 117 and 123. This allows for fluid flow between wound site 106 and removed fluid container 104 and prevents fluid flow to/from instillation fluid container 102 and filter 139. With ports 121 and 119 open, controller 130 can operate pump 110 in a second direction (e.g., clockwise in FIG. 7) to draw air into combined fluid delivery and removal line 120. The air may flow through valve 108, through removed fluid line 118, and into removed fluid container 104. Any air drawn into removed fluid container 104 can be vented from removed fluid container 104 via air vent 134. Controller 130 may continue to operate pump 110 in the second direction until the pressure measured by pressure sensor 128 (i.e., the pressure at wound site 106) reaches a negative pressure threshold (e.g., −10 mmHg). When the negative pressure threshold is reached, controller 130 may stop pump 110.

Controller 130 can count the number of rotations of pump 110 required to change the pressure at wound site 106 from the positive pressure threshold to the negative pressure threshold. Alternatively, controller 130 can determine the amount of time required to change the pressure at wound site 106 from the positive pressure threshold to the negative pressure threshold while operating pump 110 in the second direction (i.e., the pump operation time). The number of rotations of pump 110 and/or the pump operation time may be proportional to the amount of fluid removed from wound site 106. Controller 130 can use the number of rotations of pump 110 and/or the pump operation time to estimate the volume of wound site 106. For example, controller 130 can apply the number of rotations of pump 110 and/or the pump operation time as an input to a function or lookup table that relates these variables to the volume of wound site 106.

Advantageously, controller 130 can use the estimated volume of wound site 106 to determine the volume of instillation fluid 112 to be delivered during wound treatment. In some embodiments, controller 130 is configured to perform the wound volume estimation process periodically over the duration of a wound therapy period. Controller 130 can generate a trend of wound volume over time to determine whether the wound volume is increasing, decreasing, or remaining unchanged. In some embodiments, controller 130 uses the trend of wound volume over time to determine whether wound site 106 includes any areas of tissue that are difficult to reach with instillation fluid 112.

### Container Fill Level Detection

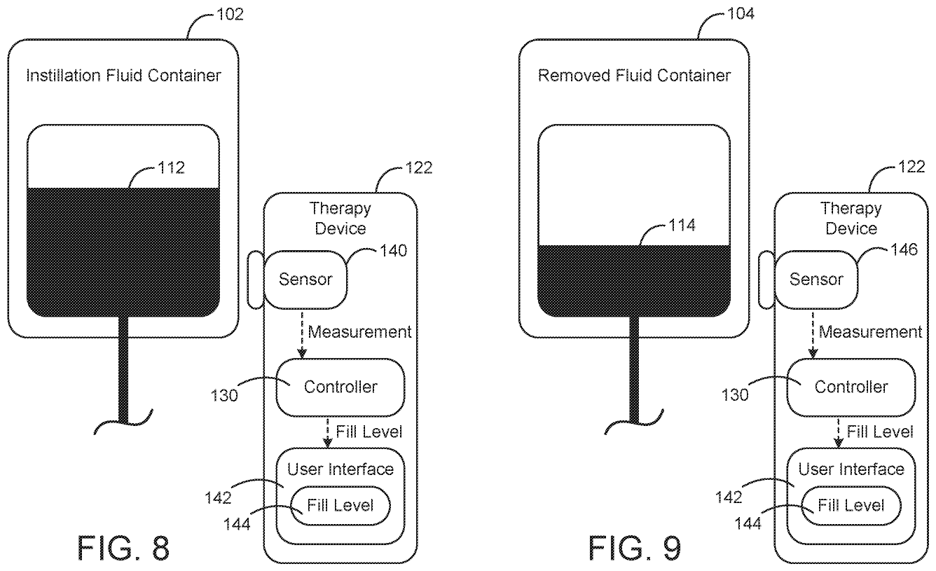

Referring now to FIGS. 8-9, block diagrams illustrating container fill level detection are shown, according to an exemplary embodiment. As shown in FIG. 8, therapy device 122 may include a sensor 140 configured to measure the amount of fluid in instillation fluid container 102. Sensor 140 may be any type of sensor configured to measure fill level or a variable related to fill level. For example, sensor 140 may be a pressure sensor configured to measure pressure at the bottom of instillation fluid container 102. The measured pressure may vary as a function of the level of instillation fluid 112 and can be used to determine the amount of instillation fluid 112 in instillation fluid container 102. Alternatively, sensor 140 may be an optical sensor, a float sensor, a weight sensor, or any other type of sensor capable of providing a measurement that indicates the amount of fluid in instillation fluid container 102.

As shown in FIG. 9, therapy device 122 may include a sensor 146 configured to measure the amount of fluid in removed fluid container 104. Sensor 146 may be any type of sensor configured to measure fill level or a variable related to fill level. For example, sensor 146 may be a pressure sensor configured to measure pressure at the bottom of removed fluid container 104. The measured pressure may vary as a function of the level of removed fluid 114 and can be used to determine the amount of removed fluid 114 in removed fluid container 104. Alternatively, sensor 146 may be an optical sensor, a float sensor, a weight sensor, or any other type of sensor capable of providing a measurement that indicates the amount of fluid in removed fluid container 104.

Controller 130 can receive measurements from sensors 140 and 146 and can use the measurements to calculate a fill level. For example, controller 130 can use the measurements from sensors 140 and 146 as inputs to a function or lookup table that relates the measured values to fill level. Controller 130 can provide an indication of the fill level 144 to a user interface 142. User interface 142 may include an electronic display, an indicator light, or other user interface element configured to provide an indication of the fill level 144. For example, user interface 142 can be configured to display a “Container Full” message when the fill level of removed fluid container 104 increases above a maximum fill level threshold. Similarly, user interface 142 can be configured to display a “Container Empty” message when the fill level of instillation fluid container 102 drops below a minimum fill level threshold.

### Therapy Device

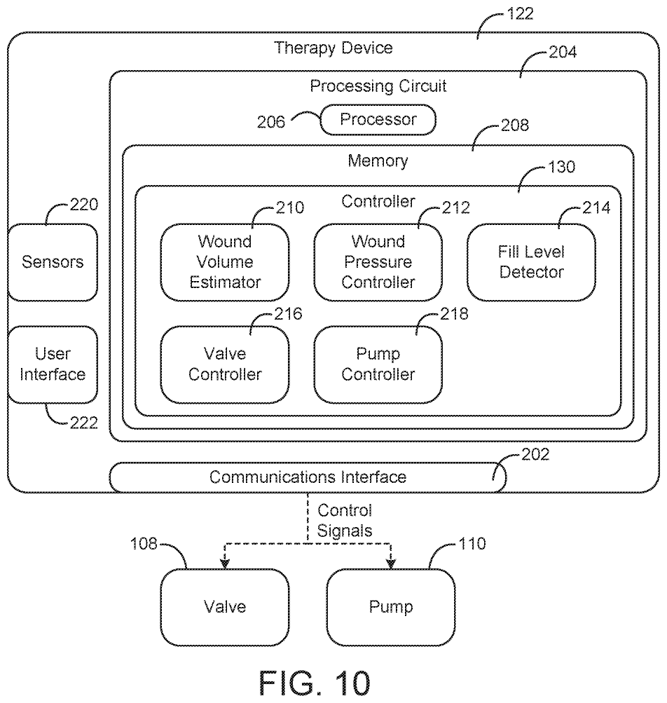

Referring now to FIG. 10, a block diagram illustrating therapy device 122 in greater detail is shown, according to an exemplary embodiment. Therapy device 122 is shown to include a communications interface 202, a processing circuit 204, sensors 220, and a user interface 222. Communications interface 202 may facilitate communications between therapy device 122 and external systems or devices. For example, communications interface 202 may receive measurements of fluid pressure at wound site 106 from pressure sensor 128. In some embodiments, communications interface 202 receives measurements from sensors 140 and 146 configured to measure the fill level of instillation fluid container 102 and removed fluid container 104. Communications interface 202 can be configured to send control signals to valve 108 and pump 110.

Communications interface 202 may include wired or wireless communications interfaces (e.g., jacks, antennas, transmitters, receivers, transceivers, wire terminals, etc.) for conducting data communications external systems or devices. In various embodiments, the communications may be direct (e.g., local wired or wireless communications) or via a communications network (e.g., a WAN, the Internet, a cellular network, etc.). For example, communications interface 202 can include an Ethernet card and port for sending and receiving data via an Ethernet-based communications link or network. In another example, communications interface 202 can include a Wi-Fi transceiver for communicating via a wireless communications network or cellular or mobile phone communications transceivers.

Processing circuit 204 is shown to include a processor 206 and memory 208. Processor 206 may be a general purpose or specific purpose processor, an application specific integrated circuit (ASIC), one or more field programmable gate arrays (FPGAs), a group of processing components, or other suitable processing components. Processor 206 is configured to execute computer code or instructions stored in memory 208 or received from other computer readable media (e.g., CDROM, network storage, a remote server, etc.).

Memory 208 may include one or more devices (e.g., memory units, memory devices, storage devices, etc.) for storing data and/or computer code for completing and/or facilitating the various processes described in the present disclosure. Memory 208 may include random access memory (RAM), read-only memory (ROM), hard drive storage, temporary storage, non-volatile memory, flash memory, optical memory, or any other suitable memory for storing software objects and/or computer instructions. Memory 208 may include database components, object code components, script components, or any other type of information structure for supporting the various activities and information structures described in the present disclosure. Memory 208 may be communicably connected to processor 206 via processing circuit 204 and may include computer code for executing (e.g., by processor 206) one or more processes described herein. When processor 206 executes instructions stored in memory 208, processor 206 generally configures therapy device 122 (and more particularly processing circuit 204) to complete such activities.

Memory 208 is shown to include controller 130. Controller 130 may include various functional modules, shown as separate components in FIG. 10. For example, controller 130 is shown to include a wound volume estimator 210, a wound pressure controller 212, a fill level detector 214, a valve controller 216, and a pump controller 218.

Wound volume estimator 210 can be configured to estimate the volume of wound site 106, as described with reference to FIGS. 6-7. For example, wound volume estimator 210 can be configured to inflate wound site 106 with air (or another fluid) until a positive pressure threshold is reached at wound site 106. Wound volume estimator 210 may receive input from sensors 220 (e.g., pressure sensor 128) to determine the pressure at wound site 106. Upon reaching the positive pressure threshold, the air (or other fluid) can be removed from wound site 106 until a negative pressure threshold is reached at wound site 106. The number of rotations of pump 110 required to move from the positive pressure threshold to the negative pressure threshold may be proportional to the volume of wound site 106. Wound volume estimator 210 may interact with valve controller 216 and pump controller 218 to send control signals to valve 108 and pump 110 during the wound volume estimation process, as described with reference to FIGS. 6-7.

Wound pressure controller 212 can be configured to regulate the pressure at wound site 106. Wound pressure controller 212 may receive input from sensors 220 (e.g., pressure sensor 128) to determine the pressure at wound site 106. Wound pressure controller 212 can be configured to operate pump 110 based on the measured pressure values. In some embodiments, wound pressure controller 212 is configured to provide negative pressure wound therapy (NPWT) by maintaining the pressure at wound site 106 at a predetermined negative pressure threshold (e.g., −10 mmHg). For example, wound pressure controller 212 can operate pump 110 to remove fluid from wound site 106 in response to a determination that the measured pressure is greater than the negative pressure threshold. By operating pump 110, fluid can be removed from wound site 106 and the pressure at wound site 106 may drop until the negative pressure threshold is reached.

In some embodiments, wound pressure controller 212 is configured to operate pump 110 in a first direction (e.g., counterclockwise in FIG. 5) to deliver instillation fluid 112 to wound site 106 in response to a determination that the measured pressure is less than a positive pressure threshold. Wound pressure controller 212 can be configured to stop operating pump 110 in the first direction in response to a determination that the measured pressure is equal to or greater than the positive pressure threshold. Similarly, wound pressure controller 212 can be configured to operate pump 110 in a second direction (e.g., clockwise in FIG. 5) to remove fluid from wound site 106 in response to a determination that the measured pressure is equal to or greater than a negative pressure threshold. Wound pressure controller 212 can be configured to stop operating pump 110 in the second direction in response to a determination that the measured pressure is less than the negative pressure threshold.

Fill level detector 214 can be configured to detect the amount of fluid in each of instillation fluid container 102 and removed fluid container 104. Fill level detector 214 can receive input from sensors 220 (e.g., sensors 140 and 146) to obtain measurements representative of the fill level of containers 102 and 104. Fill level detector 214 can use the measurements to calculate a fill level. For example, fill level detector 214 can use the measurements from sensors 220 as inputs to a function or lookup table that relates the measured values to fill level. Fill level detector 214 can provide an indication of the fill level to a user interface 222. User interface 222 may include an electronic display, an indicator light, or other user interface element configured to provide an indication of the fill level. For example, user interface 222 can be configured to display a “Container Full” message when the fill level of removed fluid container 104 increases above a maximum fill level threshold. Similarly, user interface 222 can be configured to display a “Container Empty” message when the fill level of instillation fluid container 102 drops below a minimum fill level threshold.

Valve controller 216 can be configured to generate control signals for valve 108. For example, valve controller 218 can generate control signals to open and close ports 117, 119, 121, and 123 of valve 108, as described with reference to FIGS. 6-7. Similarly, pump controller 218 can be configured to generate control signals for pump 110. For example, pump controller 218 can generate control signals to operate pump 110 in a first direction to deliver fluid to wound site 106 and control signals to operate pump 110 in a second direction to remove fluid from wound site 106. Valve controller 216 and pump controller 218 can operate in conjunction with wound volume estimator 210 and wound pressure controller 212 to carry out the wound volume estimation and pressure control processes described herein.

### Configuration of Exemplary Embodiments

The construction and arrangement of the systems and methods as shown in the various exemplary embodiments are illustrative only. Although only a few embodiments have been described in detail in this disclosure, many modifications are possible (e.g., variations in sizes, dimensions, structures, shapes and proportions of the various elements, values of parameters, mounting arrangements, use of materials, colors, orientations, etc.). For example, the position of elements can be reversed or otherwise varied and the nature or number of discrete elements or positions can be altered or varied. Accordingly, all such modifications are intended to be included within the scope of the present disclosure. The order or sequence of any process or method steps can be varied or re-sequenced according to alternative embodiments. Other substitutions, modifications, changes, and omissions can be made in the design, operating conditions and arrangement of the exemplary embodiments without departing from the scope of the present disclosure.

The present disclosure contemplates methods, systems and program products on any machine-readable media for accomplishing various operations. The embodiments of the present disclosure can be implemented using existing computer processors, or by a special purpose computer processor for an appropriate system, incorporated for this or another purpose, or by a hardwired system. Embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. Combinations of the above are also included within the scope of machine-readable media. Machine-executable instructions include, for example, instructions and data which cause a general purpose computer, special purpose computer, or special purpose processing machines to perform a certain function or group of functions.

Although the figures show a specific order of method steps, the order of the steps may differ from what is depicted. Also two or more steps can be performed concurrently or with partial concurrence. Such variation will depend on the software and hardware systems chosen and on designer choice. All such variations are within the scope of the disclosure. Likewise, software implementations could be accomplished with standard programming techniques with rule based logic and other logic to accomplish the various connection steps, processing steps, comparison steps and decision steps.

1. A wound therapy system for delivering fluid to a wound site and removing fluid from the wound site, the wound therapy system comprising:
an instillation fluid container configured to store instillation fluid for delivery to the wound site;
a removed fluid container configured to store fluid removed from the wound site;
a combined fluid delivery and removal line fluidly coupled to the wound site, the instillation fluid container, and the removed fluid container; and
a single pump coupled to the combined fluid delivery and removal line and configured to:
operate in a first direction to draw the instillation fluid from the instillation fluid container and deliver the instillation fluid to the wound site via the combined fluid delivery and removal line; and
operate in a second direction opposite the first direction to remove fluid from the wound site and deliver the removed fluid to the removed fluid container via the combined fluid delivery and removal line.

2. The wound therapy system of claim 1, wherein the pump is a peristaltic pump and the combined fluid delivery and removal line passes through the peristaltic pump.

3. The wound therapy system of claim 1, further comprising a three-way valve fluidly coupled to the instillation fluid container, the removed fluid container, and the combined fluid delivery and removal line.

4. The wound therapy system of claim 3, wherein the three-way valve is configured to:
direct fluid flow from the instillation fluid container to the combined fluid delivery and removal line when the pump is operated in the first direction; and
direct fluid flow from the combined fluid delivery and removal line to the removed fluid container when the pump is operated in the second direction.

5. (canceled)

6. (canceled)

7. (canceled)

8. The wound therapy system of claim 1, further comprising:
an instillation fluid line connecting the instillation fluid container to the combined fluid delivery and removal line;
a one-way valve disposed along the instillation fluid line and configured to allow fluid flow from the instillation fluid container to the combined fluid delivery and removal line and prevent fluid flow from the combined fluid delivery and removal line to the instillation fluid container;
a removed fluid line connecting the removed fluid container to the combined fluid delivery and removal line; and
a one-way valve disposed along the removed fluid line and configured to allow fluid flow from the combined fluid delivery and removal line to the removed fluid container and prevent fluid flow from the removed fluid container to the combined fluid delivery and removal line.

9. (canceled)

10. The wound therapy system of claim 1, further comprising:
a pressure sensor configured to measure pressure at the wound site; and
a controller configured to operate the pump based on the measured pressure;
wherein the controller is configured to:
monitor the measured pressure at the wound site; and
operate the pump to maintain the measured pressure at a predetermined pressure threshold.

11. (canceled)

12. The wound therapy system of claim 10, wherein the controller is configured to:
monitor the measured pressure at the wound site;
operate the pump in the first direction to deliver the instillation fluid to the wound site in response to a determination that the measured pressure is less than a positive pressure threshold;
stop operating the pump in the first direction in response to a determination that the measured pressure is equal to or greater than the positive pressure threshold;
operate the pump in the second direction to remove fluid from the wound site in response to a determination that the measured pressure is equal to or greater than a negative pressure threshold; and
stop operating the pump in the second direction in response to a determination that the measured pressure is less than the negative pressure threshold.

13. (canceled)

14. The wound therapy system of claim 10, wherein the controller is configured to estimate a volume of the wound site by:
counting a number of rotations of the pump required to change the measured pressure by a predetermined amount;
estimating the volume of the wound site based on the counted number of rotations;
wherein counting the number of rotations of the pump required to change the measured pressure by a predetermined amount comprises:
operating the pump in the first direction until the measured pressure reaches a positive pressure threshold;
operating the pump in the second direction until the measured pressure reaches a negative pressure threshold; and
counting the number of rotations of the pump required to change the measured pressure from the positive pressure threshold to the negative pressure threshold.

15. (canceled)

16. A wound therapy system for delivering fluid to a wound site and removing fluid from the wound site, the wound therapy system comprising:
a combined fluid delivery and removal line fluidly coupled to the wound site; and
a peristaltic pump coupled to the combined fluid delivery and removal line and configured to:
operate in a first direction to deliver instillation fluid to the wound site via the combined fluid delivery and removal line; and
operate in a second direction opposite the first direction to remove fluid from the wound site via the combined fluid delivery and removal line.

17. The wound therapy system of claim 16, further comprising an instillation fluid container configured to store the instillation fluid for delivery to the wound site and a removed fluid container configured to store the fluid removed from the wound site;
wherein the peristaltic pump is configured to draw the instillation fluid from the instillation fluid container and deliver the instillation fluid to the wound site via the combined fluid delivery and removal line when the peristaltic pump is operated in the first direction; and
wherein the peristaltic pump is configured to draw the removed fluid from the wound site and deliver the removed fluid to the removed fluid container via the combined fluid delivery and removal line when the peristaltic pump is operated in the second direction.

18. The wound therapy system of claim 17, further comprising:
an instillation fluid line connecting the instillation fluid container and the combined fluid delivery and removal line; and
a first one-way valve disposed along the instillation fluid line and configured to allow fluid flow from the instillation fluid container to the combined fluid delivery and removal line and prevent fluid flow from the combined fluid delivery and removal line to the instillation fluid container;
a removed fluid line connecting the removed fluid container and the combined fluid delivery and removal line; and
a second one-way valve disposed along the removed fluid line and configured to allow fluid flow from the combined fluid delivery and removal line to the removed fluid container and prevent fluid flow from the removed fluid container to the combined fluid delivery and removal line.

19. (canceled)

20. (canceled)

21. The wound therapy system of claim 16, further comprising:
an instillation fluid container;
a removed fluid container;
a three-way valve fluidly coupled to the instillation fluid container, the removed fluid container, and the combined fluid delivery and removal line;
wherein the three-way valve is configured to:
direct fluid flow from the instillation fluid container to the combined fluid delivery and removal line when the peristaltic pump is operated in the first direction; and
direct fluid flow from the combined fluid delivery and removal line to the removed fluid container when the peristaltic pump is operated in the second direction.

22. (canceled)

23. (canceled)

24. (canceled)

25. (canceled)

26. The wound therapy system of claim 16, further comprising:
a pressure sensor configured to measure pressure at the wound site;
a controller configured to operate the peristaltic pump based on the measured pressure;
wherein the controller is configured to:
monitor the measured pressure at the wound site; and
operate the peristaltic pump to maintain the measured pressure at a predetermined pressure threshold.

27. (canceled)

28. (canceled)

29. (canceled)

30. (canceled)

31. (canceled)

32. A method for delivering fluid to a wound site and removing fluid from the wound site, the method comprising:
fluidly coupling a combined fluid delivery and removal line to the wound site;
operating a peristaltic pump in a first direction to deliver instillation fluid to the wound site via the combined fluid delivery and removal line; and
operating the peristaltic pump in a second direction opposite the first direction to remove fluid from the wound site via the combined fluid delivery and removal line.

33. (canceled)

34. (canceled)

35. (canceled)

36. (canceled)

37. The method of claim 32, further comprising using a three-way valve to control fluid flow among an instillation fluid container, a removed fluid container, and the combined fluid delivery and removal line.

38. (canceled)

39. (canceled)

40. (canceled)

41. The method of claim 32, further comprising:
measuring a pressure at the wound site; and
operating the peristaltic pump based on the measured pressure;
wherein operating the peristaltic pump based on the measured pressure comprises operating the peristaltic pump to maintain the measured pressure at a predetermined pressure threshold.

42. (canceled)

43. The method of claim 41, wherein operating the peristaltic pump based on the measured pressure comprises:
operating the peristaltic pump in the first direction to deliver the instillation fluid to the wound site in response to a determination that the measured pressure is less than a positive pressure threshold; and
stopping operating of the peristaltic pump in the first direction in response to a determination that the measured pressure is greater than or equal to the positive pressure threshold.

44. The method of claim 41, wherein operating the peristaltic pump based on the measured pressure comprises:
operating the peristaltic pump in the second direction to remove fluid from the wound site in response to a determination that the measured pressure is greater than or equal to a negative pressure threshold; and
stopping operation of the peristaltic pump in the second direction in response to a determination that the measured pressure is less than the negative pressure threshold.

45. The method of claim 41, further comprising:
counting a number of rotations of the peristaltic pump required to change the measured pressure by a predetermined amount; and
estimating the volume of the wound site based on the counted number of rotations.

46. The method of claim 45, wherein counting the number of rotations of the peristaltic pump required to change the measured pressure by a predetermined amount comprises:
operating the peristaltic pump in the first direction until the measured pressure reaches a positive pressure threshold;
operating the peristaltic pump in the second direction until the measured pressure reaches a negative pressure threshold; and
counting the number of rotations of the peristaltic pump required to change the measured pressure from the positive pressure threshold to the negative pressure threshold.
